(19) United States
(12) Patent Application Publication
(10) Pub. No.: US 2006/0219251 A1
(43) Pub. Date: Oct. 5, 2006

(54) System and method for deconditioning habitual nail biting

(76) Inventor: Charles D. Ray, Santa Barbara, CA (US)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 11/398,007

(22) Filed: Apr. 5, 2006

Related U.S. Application Data
(60) Provisional application No. 60/668,343, filed on Apr. 5, 2005.

(51) Int. Cl. A61F 5/37

(52) U.S. Cl. 128/880

(57) ABSTRACT

A system used in the deconditioning of compulsive fingernail biting includes motivating behavior at timed intervals. A method of negative feedback using positive responses is in most cases is curative of the compulsion. The timing interval between behavior motivation sessions can be adjusted for best effects in a given habitué. One version has no electronics and is used as a decoy finger for biting thus sparing the natural ones.

### CROSS-SECTION TO RELATED APPLICATIONS

This application claims priority to U.S. Provisional Application Ser. No. 60/668,343, filed Apr. 5, 2005, the teachings of which are incorporated herein by reference in their entirety.

### FIELD OF THE INVENTION

The present invention relates to personal health and hygiene. More specifically, the present invention relates to systems and methods for reducing or stopping the undesirable behavior of nail biting.

### BACKGROUND

Behaviors that compel individuals to act in certain ways are essential to life and social participation. The need to sleep, eat, bathe, dress and attend work or school at certain hours is essential for normal growth and success in life. Individuals may not want these things to become needed, but various pressures require them. However, certain compelling behavior, obsessive-compulsions or habits, almost always learned earlier in life, may be a nuisance and interference with preferred or normal behavior in later childhood or adult life. It is estimated that as much as 2% of our population has undesirable compulsive habits, where women are slightly more likely to be affected than men. When severe, such disorders become a form of mental illness where the person seems possessed by intrusive thoughts (called obsessions) or feels bound to repetitive performance of behavior patterns (compulsions) and is unable to control them. Like all psychological conditions, mild and severe, all humans experience elements of borderline or frank mental aberrations at times, usually triggered by unusual circumstances like severe stress. On occasion the undesirable ideation or performance persists to become a true abnormality where the person finds the disturbing thoughts or actions interfere with normal daily functions of social interaction, employment, outside activities or schoolwork.

Although a great number of such persons realize their affliction, they are powerless to deal with them. This is generally quite unlike the severe mental disorders where the person is unaware of, or lacks insight into, the problem, thereby interfering with possible treatment. To the compelled person the behavior is primarily a secret ritual that begs or demands attention and satisfaction, often on a constant basis that may interfere with normal behavior or have undesirable outcomes.

Treatments for the more difficult forms of these disorders rely on psychotherapy, medication, or both. Behavior modification is quite important and is employed in a variety of ways, such as response intervention—where the precipitating thoughts, circumstances or influences are artificially presented and the patient is protected or withdrawn from the threat, instantly altering his ordinary compelled reaction. As one example, computer generated artificial reality employs this dramatic method. Another form of exposure-prevention would be to present a precipitating situation and then disallow the patient from performing his usual compelled behavior. This creates anxiety and tension against which the therapist then works. Such methods are prolonged and often complex but may be quite successful. Habits are generally milder but may be indistinguishable from compulsions.

Habits also are acts arising from experience and are generally regularly and automatically performed. They include repetitive mannerisms such as motions of face, hands and limbs, but also cravings such as smoking or overeating, characteristic reading of selected topics such as magazines or pornography, motion picture attendance, video game playing or watching certain TV performances. Their basis is learned and represents problems needing treatment when they disrupt a person's well-being or preferred social interactions. Classical Freudian psychoanalysts considered habits as expressions of erotic and aggressive impulses, as they did much abnormal behavior. Clearly, when repressed, such impulses may find outlets through other counterproductive behaviors including habit formation. Much more practically, the evidence is less theoretical and indicates that habituation is a matter of learned response conditioning and reinforcement over which the person has little control and is usually sustained by circumstances or cues that are quite different from those upon which the habit was initially established. Indeed, any action, good or bad, that is well rewarded is likely to become habituating, yielding its own reward in time.

Importantly, most habits have components of which the habitué may be quite unaware. The habituated person is aware of the outcome of this undesirable, even potentially dangerous problem, but the actual performance is usually beneath their consciousness. When habitués are unaware of and thus do not suppress the importance of the acts, they do not go away. An important method for eliminating conditioned habits and compulsions is that of counter conditioning or deconditioning or awareness recognition. These can be performed successfully in two ways: (1) the reward aspect is purposely thwarted by giving a bad or absent reward which helps to extinguish the behavior, or (2) the regular occurrence of the habit is forced into being a conscious act over which the individual can then exert a controlling influence.

Habitual biting of nails is one type of conditional habit or compulsion, which can cause serious infections, deformity and cosmetically undesirable changes in otherwise normal fingers. Such impaired fingers and nails may interfere with occupational activities as well. Habitual biters are almost universally ashamed of their condition but are generally unable to stop without assistance. A common method to discourage nail biting is to apply artificial nails, which when decorated can be expensive such that a financial incentive not to destroy the nails exists. When severe and unalterable, extended psychotherapy, medication and behavioral modification have been successful in treating this disorder from becoming a potential mental illness. However, such techniques can be costly. Counter conditioning or deconditioning nail biting may involve a negative or punitive reward such as blaming or punishing, coating the involved fingers with evil tasting or blistering materials, or electric shocks to the mouth in contact with the fingers. These methods have been shown to have even more undesirable side effects in some persons, especially children, commonly resulting in a refusal to use the device. As such, a need exists for a new approach to the deconditioning of undesirable behavior, such as nail biting.

### SUMMARY

One aspect of the present invention relates to treating the habit of compulsive fingernail biting by establishing an awareness of the act, thereby rendering the performance a conscious act. The act then no longer is unconsciously operating and a positive competitive act is added. In one embodiment, the positive competitive act results from biting an artificial nail or biting an end of a prosthesis body to turn on or off an alarm or other prompting device. This act is closely akin to classical Pavlovian conditioning.

By making the act conscious, the individual learns to control it himself within time. In another aspect of the present invention, the nail biting and prompting device, such as an alarm, should be paired to accelerate a deconditioning process, but not at the expense of deformation of natural fingers. In one embodiment, no punitive negative reward is given, such as the pain of biting the “quick” of a nail. Instead, a positive competitive act, such as switching off of a prompting device, such as a barely perceptible, but annoying alarm is achieved by biting an artificial nail, or otherwise actuating a switch.

Actually removing substance by biting the artificial nail is not necessary, but means for such is incorporated into some embodiments of the present invention. In embodiments without such means, it is only the act of commencing to bite that counts. Another aspect of the present invention relates to reasonable social and occupational acceptance, wherein the artificial finger not only has a reasonable look and feel of a natural finger, but also allows performance of occupational tasks like keyboard typing, operating button-controlled devices, eating, dressing and other acts of daily living.

In this respect, embodiments of the invention are designed to provide adequate aesthetic appearance of a relatively normal, although shorter, finger firmly strapped to the hand between the thumb and index fingers. This position provides needed physical and tactile feedback to the habitué as though a normal finger and a rewarding sensation as though biting a normal nail while the act is being deconditioned. In one embodiment, the comforting components of the artificial finger are then rudely interrupted by a tactile alarm that is not externally obtrusive. This alarm can be stopped by biting the artificial nail or the buried switch in the artificial finger's tip, turning the device off until the next preset time.

Other aspects of the present invention relate to a prosthesis assembly configured to be strapped into a space between a thumb and an index finger and from there tightly around a wrist. In one embodiment, the prosthesis body is shorter than either natural finger. In some embodiments, the prosthesis body is waterproof, sanitary, and/or easily cleaned. In some embodiments, acrylic or polyethylene or similar polymer nails are replaceably employed, with or without flavors applied. In other embodiments, a simple disc silver oxide battery is used with a two-part alarm consisting of a gentle piezoelectric vibrator and a visible LED indicator. A pressure-operated switch placed near the biting end is gently bitten to turn the alarm off. A second pressure-operated switch operates the contained waterproof replaceable battery circuit. In some embodiments, the prosthesis body or finger has a molded flesh colored outer washable shell of a soft skin-like vinyl, polypropylene or silicone rubber to which adjustable self fixating flesh colored hook and loop straps provide stability of the novel device when removably attached to either right or left hand and wrist.

Other aspects of the present invention relate to a method of deconditioning. In one embodiment, when the habitué is biting the replaceable artificial nail, the prosthesis body lies inside the two natural fingers, which can then firmly touch a face of a user around a mouth of the user further giving a feeling of normal anatomical and mechanical feedback and a familiar comfort while nail biting. The system is applied at any conscious time, when at leisure or avocational activities. In one method of deconditioning, the prosthesis is worn during a training cycle to learn its proper operation.

### BRIEF DESCRIPTION OF THE DRAWINGS

Embodiments of the invention will be described with respect to the figures, in which like reference numerals denote like elements, and in which:

FIG. 1 is a perspective view of a deconditioning system according to principles of the present invention.

FIG. 2 is a plan view of a prosthesis assembly of the deconditioning system of FIG. 1.

FIG. 3A is a cross-sectional view of the assembly of FIG. 2, taken along the lines A-A.

FIG. 3B is a cross-sectional view of the assembly of FIG. 2, taken along the lines B-B.

FIG. 3C is a cross-sectional view of the assembly of FIG. 2, taken along the lines C-C.

FIG. 3D is a cross-sectional view of the assembly of FIG. 2, taken along the lines D-D.

FIG. 4 is a circuit diagram illustrating one embodiment of deconditioning circuitry useful with the deconditioning system of FIG. 1.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “dorsum,” “back,” “palmar,” “front,” “tip,” or “distal,” “base,” “proximal,” etc. is used with reference to the orientation of the Figure(s) being described. Because components of embodiments of the present invention can be positioned and/or operated in a number of different ways, structural or logical changes may be made without departing from the scope of the present invention. The following detailed description, therefore, is not to be taken in a limiting sense, and the scope of the present invention is defined by the appended claims.

Referring now to the drawings which are not to scale and which illustrate preferred forms of embodiments in accordance with the present invention. FIG. 1 shows a perspective view of a deconditioning system 10 in accordance with the present invention applied to a user's hand 12. Details on various components of the deconditioning system 10 are provided below. In general terms, however, the system 10 includes a prosthesis assembly 14 and an attachment device 16. The prosthesis assembly 14 includes a prosthesis body 18 housing or maintaining various components described below, and defining a base 20 to which the attachment device 16 is secured. With this in mind, the user's hand 12 is spread open with the deconditioning prosthesis body 18 attached thereto. The hand 12 includes a thumb 22 and an index finger 24. As shown, the prosthesis body 18 is positioned in an anatomical valley defined between the thumb 22 and the index finger 24. The attachment device 16 is provided for attaching the prosthesis body 18 to the hand 12. As shown in one embodiment, the attachment device 16 comprises adjustable strap(s) including hook and loop fasteners (not shown) forming part of the palmar extensions of the straps and configured to permit firm positioning and easy removal of the prosthesis assembly 14.

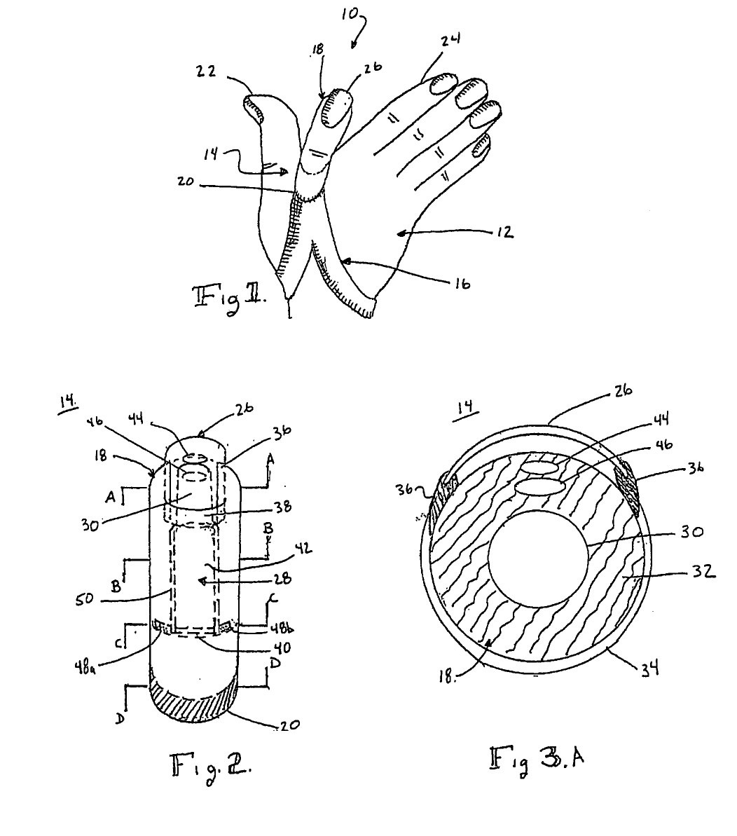

FIG. 2 is plan view of the prosthesis assembly 14. In general terms, the prosthesis assembly 14 includes the prosthesis body 18, an artificial fingernail 26, and deconditioning circuitry 28 (referenced generally). The artificial fingernail 26 is mounted to, and extends exteriorly from, the prosthesis body 18. The deconditioning circuitry 28 is disposed within the prosthesis body 18 and, as described below, is adapted to perform one or more deconditioning operations.

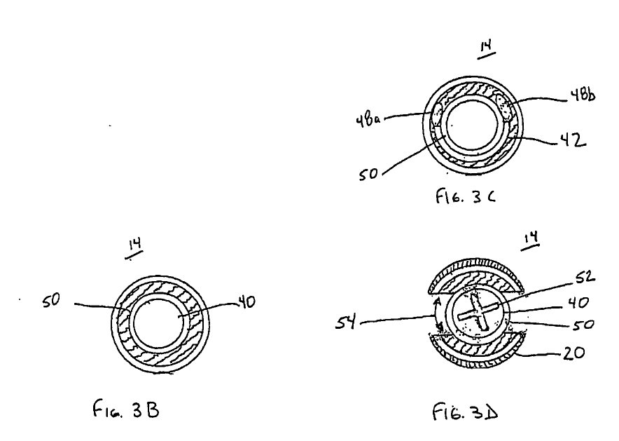

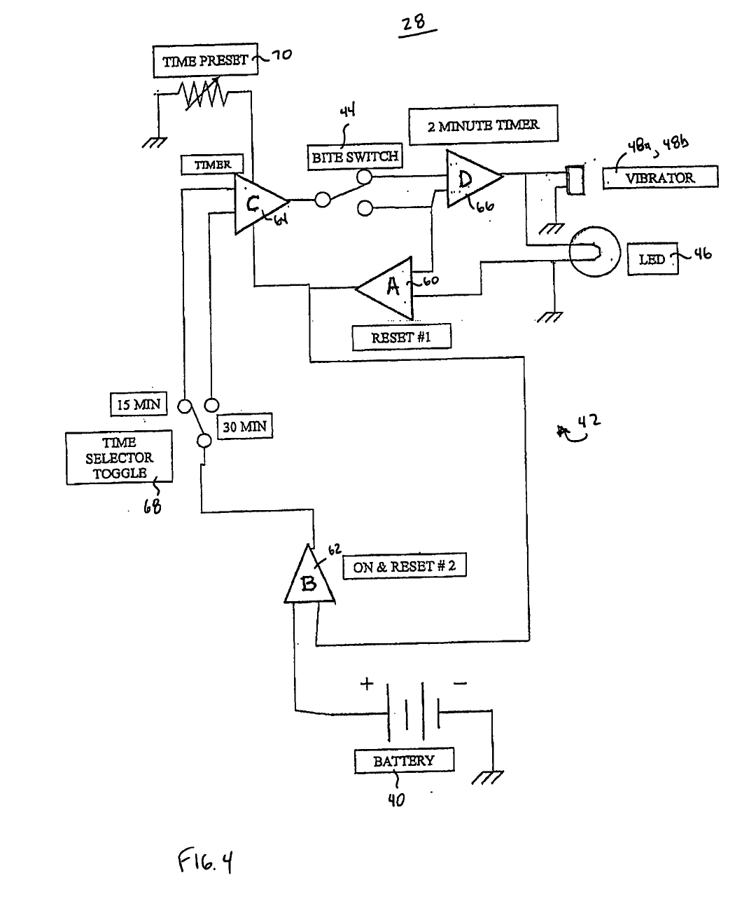

In one embodiment, the prosthesis body 18 defines a physical texture and structural aesthetics resembling a normal human finger, and includes various structures within the prosthesis body 18 for maintaining other components of the prosthesis assembly 14. An internal structure of the prosthesis body 18 has an extension 30 of firm material imitating a natural subsurface soft bone of a natural human finger. With additional reference to FIG. 3A, a predominate inside mass 32 of the prosthesis body 18 is made, in one embodiment, of molded, semi-firm elastomers, such as polyurethane. In some embodiments, an outer skin material 34 is applied over the mass 32 and provides the prosthesis body 18 with a more natural looking appearance and can have a desired color.

With continued reference to FIGS. 2 and 3A, in one embodiment, the artificial fingernail 26 is removably maintained by the prosthesis body 18 by sliding of the artificial fingernail 26 edges into channels 36 defined by the prosthesis body 18 until the fingernail 26 firmly engages a retaining clip 38, this clip 38 being made integral with the two side guide channels 36.

The deconditioning circuitry 28 can assume a wide variety of forms, and in one embodiment, includes a power source (e.g., battery) 40, circuitry 42 (referenced generally in FIG. 2), a switch 44, and one or more prompting devices, for example the prompting devices 46 and 48a, 48b. As described below, the power source 40 powers the components 42-48b, with the circuitry 42 controlling operation of the prompting devices 46, 48a, 48b, based, in some embodiments, upon signals received from the switch 44. The switch 44 is a “bite” switch, adapted to effectuate a change in state (e.g., on-to-off or off-to-on) when subjected to a certain level of pressure or compression indicative of a biting force generated by a human jaw. The prompting devices 46, 48a, 48b can assume a variety of forms including, for example, lights, vibrators, audible alarms, etc. In one embodiment, the prompting device 46 is an LED light and the prompting devices 48a, 48b are sonic vibrators. In other embodiments, an audible alarm device (not shown) is further provided. Alternatively, however, only one of the prompting devices 46, 48a, and/or 48b need be included.

With the above general explanation of the deconditioning circuitry 28 in mind, in one embodiment, the compression ‘bite’ switch 44 is imbedded within the prosthetic body 18 near the outermost portion of the fingernail 26. In one embodiment, the imbedded LED lamp 46 is visible through the translucent, replaceable polymeric fingernail 26. An inside pocket holds the replaceable battery 40 having a removable end cap. The sonic vibrators 48a, 48b lie near a battery compartment wall 50 in position at the end of the prosthesis body 18 and, upon assembly to the user's hand 12, abut the valley defined between the thumb finger 22 and the index finger 24 (FIG. 1). Thus, in one embodiment, the bite switch 44 and indicator LED 46 are located near the distal tip of the prosthesis body 18, near a tip of a nail bed of the prosthesis body 18.

FIG. 3B is a cross-sectional view approximately mid-body of the prosthesis body 18 showing the battery 40 and the surrounding battery compartment wall 50. FIG. 3C is a cross-sectional view near the lower, proximal end of the prosthesis body 18 illustrating the plurality of vibratory transducers of the sonic vibrators 48a, 48b in close proximity to the battery compartment wall 50, as well as the circuitry 42. The circuitry 42 is described in greater detail below, but is generally formed as a flex circuit (e.g., on a flexible substrate) and assembled to the prosthesis body 18 in the curved manner shown in FIG. 3C.

FIG. 3D is a cross-sectional view at the base 20 of the prosthesis body 18 as it sits on the skin web fold between the thumb 22 (FIG. 1) and index finger 24 (FIG. 1). The battery compartment wall 50 and the removable watertight battery cap having a Philips-head slot 52 (in accordance with one embodiment) for easy removal are also illustrated. In one embodiment, a gap 54 is defined by the base 20. The gap 54 allows the base 20 to straddle the skin web between the thumb 22 and the index finger 24.

FIG. 4 is a circuit diagram of a one of several possible configurations of the deconditioning circuitry 28, and diagrammatically represents the battery 40, the circuitry 42, the switch 44, and the prompting devices 46, 48a, 48b. The circuitry or circuit board 42 includes, in one embodiment, first and second resets 60, 62 (e.g., op-amp A and op-amp B), first and second timers 64, 66 (e.g., op-amp C and op-amp D), and a time selector switch 68. The relationships and interaction between these components are described below. However, in other embodiments, a variety of other possible circuits can be employed, capable of achieving similar functions as the circuit board 42 shown.

In one embodiment, the system 10 remains ‘off’ until the bite switch 44 at the distal end of the prosthesis 18 (FIG. 1) is compressed by biting the prosthesis body's tip or the distal end of the artificial nail 26 (FIG. 2). This action energizes op-amps D and A, which then energizes op-amp B. An output pulse passes through the time selector toggle 68 triggering either of two timing interval conditions for op-amp C. The interval selected, starting at either 15 or 30 minutes, is alterable by a time preset control 70, the access to which is provided on the circuit board 42 (FIG. 3C).

The trigger output from op-amp C then sends a single or dual pulse, depending on the shorter or longer interval selected through the op-amp D simultaneously into the vibrators 48a, 48b and LED 46 to confirm whether the interval is the shortest or longest selected by a wearer otherwise operating the time selector toggle switch 68.

In one embodiment, these pulses last only two seconds and are perceived as light from the wearer by the LED 46 visible through the artificial fingernail 26 (FIG. 2) and the perceived through tingling from the vibrator(s) 48a, 48b. At the end of the preset interval (15 or 30 minutes or other intervals as preset), a behavior motivator such as an audible, visual, or tactile alarm or combinations thereof is activated. In one embodiment, ‘alarm pulse(s)’ alerts the wearer to bite the fingernail bite switch 44 on command. Alarm amplitude can be set so as not to be noticeable by an outside person. If after the alarm pulses, a responsive bite is not received within 2 minutes, the system 10 shuts down to conserve battery power and remains off until restarted again by a new bite.

In one embodiment of a deconditioning method and with reference to FIGS. 1-4, the cleaned and sanitized deconditioning device is tested for battery power by compressing or biting the bite switch 44 and observing the brief pulse(s) of the LED 46 light in the finger's tip. This confirms that the battery 40 has sufficient reserve and which of the timing intervals has been set, there being one for the shorter period and two or more for longer ones. The prosthesis body 18 is then strapped to the hand 12 using attached, flesh-tinted stabilizing straps 16 having hook and loop fastener ends (or other attachment devices). The fit should be snug and stable; the user having been instructed to seek an optimal position and tightness. Of course, when occupied with activities that would be contrary to the use of the device, such as bathing or washing dishes or working in wet or greasy unclean environments, the prosthesis body 2 should not be applied or should be taken off and protected.

When in position, the bite switch 44 is again engaged and usage begins. At each adjustable selected interval, such as every 15 or 30 minutes or other preselected intervals, the gentle tactile alarm perceptibly vibrates and the visible, faint LED light 46 illuminates for timed nail biting to take place.

Biting or chewing on the artificial nail 26 or nibbling on the end of the prosthetic body 18 shuts off the alarm. If within 2 minutes (or some other desired interval) no response is given, the electronic circuit 28 shuts off. The user will accommodate to this routine quickly. After approximately one to three weeks of training, the prosthesis body 18 can be left off for a day or so to see if the deconditioning is progressing. When no inadvertent biting occurs during that test period, the rest period may be extended.

However, it the unconscious biting returns, or the individual feels an anxious need to bite, the prosthesis body 18 is returned to the hand 12. After prolonged successful abstinence from the use of the deconditioning system 10, with amelioration of the problem, the use of the deconditioning system 10 may be discontinued to be used again if the habit were to reemerge under stressful or other circumstances.

Routing daily maintenance consists of gently washing and drying the prosthesis body 18 and straps 16 while off the hand 12. When the deconditioning system 10 has not had a bite within a certain period of time (e.g., 2 hours), the circuit automatically shuts off to preserve battery life. Access to adjustments and battery replacement is realized through an openable port in the base 20 of the prosthesis body 18, away from the nail 26 and opposite end of the prosthesis body 18. The entire prosthesis body 18, and attachment device, or straps 16, is water and saliva proof and easily sanitized. When the artificial nail 26 needs to be replaced, after it shows wear or is ragged, a new artificial nail 26 adapted from standard commercial artificial nails, trimmed by a pre-shaped clip unit provided to the wearer, readies the replacement to be attached. The old nail 26 is pulled out and the new one is slid onto the prosthesis body 18 along the side channels 36 to be held in position by the retaining friction clip 38 constructed, for example, of stainless steel and located at the nail 26 base upon final assembly. The retaining clip 38 is, in one embodiment, fabricated together with the side nail guides 36.

Thus, the two features 36 serve together to guide and stably but removably retain the nail 26 inside a dorsum of the prosthesis body 18, near the position analogous to a natural nail base.

Some advantages that can be achieved in some embodiments of the present invention include a socially and cosmetically acceptable, removable, adjustable and replenishable device that has the ability to eliminate an annoying and potentially dangerous habit using a gentle, negative feedback process that is positively rewarding. Cosmetic, tactile and positioning senses give the user a readily acceptable sensory substitute for a natural finger while a built-in structured deconditioning program continues, all of which is determined by simple electronics contained within the prosthesis body 18. This device can accomplish what could otherwise be a far lengthier and expensive treatment program including medication and psychotherapy or behavioral modification. The construction of the deconditioning device is relatively simple and sanitary and should be inexpensive to manufacture in quantity from standard parts openly available. The waterproof outer ‘skin’ 34 not only can be easily sanitized but can also be replaced if needed or if the device is shared among family members. Further the ‘skin’ 34 can include various colors to suit racial color needs.

In one embodiment, the deconditioning system 10 is used in the process of behavioral modification by the conscious application to the essentially unconscious habituation of undesirable behavior, such as fingernail biting. Embodiments of the deconditioning system 10 can also be made in a simpler, totally passive form, without the electronics or indication components as described. In these embodiments, the passive system or device serves to give the somewhat less addicted habitué a tangible means to satisfy the urge, but on an inanimate object, thereby sparing the habitué's natural fingers and nails. In another embodiment, the electronics consist of a simple preset time alarm and an on-off switch, where the responses to the alarm do not operate as a closed-loop feedback system, but an open-loop response to the timing stimulus.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A deconditioning system for stopping or reducing undesirable behavior comprising:
a prosthesis assembly including a prosthesis body maintaining a prompting device electronically connected to a switch; and
an attachment device coupled to the prosthesis body and configured for attaching the prosthesis assembly to a user.

2. The deconditioning system of claim 1, wherein the undesirable behavior is nail biting and the prosthesis body is configured to resemble a human finger.

3. The deconditioning system of claim 2, further including an artificial nail coupled to the prosthesis body.

4. The deconditioning system of claim 3, wherein the switch is positioned to be actuated when a user bites the artificial nail.

5. The deconditioning system of claim 2, wherein the prosthesis body and attachment device are configured to removably affix the prosthesis body in a position between a thumb and an index of the user.

6. The deconditioning system of claim 2, wherein the attachment device includes an elastic strap.

7. The deconditioning system of claim 2, wherein the prosthesis body includes an outer skin configured to resemble a look and feel of a human finger.

8. The deconditioning system of claim 1, wherein the device further comprises:
a programmable timing mechanism electronically connected to the prompting device, wherein the programmable timing mechanism is configured to effectuate performance of a deconditioning program.

9. The deconditioning system of claim 8, wherein the programmable timing mechanism includes circuitry adapted to operate the prompting device in response to signals from the switch.

10. The deconditioning system of claim 1, wherein the prosthesis assembly includes a timer for controlling operation of the prompting device.

11. The deconditioning system of claim 1, wherein the prompting device includes a vibrator.

12. The deconditioning system of claim 1, wherein the prompting device includes an audible alarm.

13. The deconditioning system of claim 1, wherein the prompting device includes a visual indicator.

14. A method of deconditioning undesirable behavior comprising:
attaching a prosthesis body to a user, wherein the prosthesis body is configured to resemble a part of the human body;
activating a prompting device carried by the prosthesis body to prompt a user behavior; and
deactivating the prompting device based upon information indicative of the user performing the behavior.

15. The method of claim 14, wherein the prosthesis body is configured to resemble a human finger and the undesirable behavior is nail biting.

16. The method of claim 14, wherein deactivating the prompting device includes biting the prosthesis body to actuate a switch electronically connected to the prompting device.

17. The method of claim 14, wherein deactivating the prompting device includes the user biting an artificial nail carried by the prosthesis body.

18. The method of claim 14, wherein the prompting device includes an audible alarm.

19. The method of claim 14, further comprising:
activating the prompting device at pre-determined intervals.
